(19) United States
(12) Patent Application Publication
(10) Pub. No.: US 2002/0051796 A1
(43) Pub. Date: May 2, 2002

(54) SOLUTION OF A POLYMER OF THE POLYACRYLIC AND/OR POLYVINYLIC TYPE ASSOCIATED WITH A FILLER AND A KERATOLYTIC AGENT, AND A COSMETIC DEVICE FOR CLEANING AND CARE OF THE SKIN

(76) Inventor: JEAN-LOUIS GUERET, PARIS (FR)

Correspondence Address: NIXON & VANDERHYE PC, 1100 NORTH GLEBE ROAD 8TH FLOOR, ARLINGTON, VA, 222014714

(21) Appl. No.: 09/461,375

(22) Filed: Dec. 15, 1999

(30) Foreign Application Priority Data
Dec. 18, 1998 (FR) 98 16019

(51) Int. Cl. A61K 6/00; A61K 7/00; A61K 9/70; A61F 13/00; A61L 15/16; A61K 9/14; A61K 47/30; A61K 47/32; A61K 47/34

(52) U.S. Cl. 424/401; 424/443; 424/447; 424/449; 424/484; 424/486; 424/487; 514/772.1; 514/772.2; 514/772.3; 514/772.4; 514/844; 514/846; 514/845; 514/847; 514/848

(57) ABSTRACT

Colorless solution for cosmetic use, based on a hydrophobic polymer in an organic solvent medium. This solution contains, in the cosmetically acceptable organic solvent, (1) at least one self-adhesive hydrophobic polymer selected from polymers or copolymers of the polyacrylic and/or polyvinylic type, (2) at least one filler and/or essential oil, and (3) at least one keratolytic agent, said polymer having an adhesiveness of between 150 and 800 g/cm² after evaporation of said organic solvent.

Use in a cosmetic device for instantaneous cleansing and care of the skin.

The present invention relates to a colorless solution for cosmetic use which contains, in a cosmetically acceptable organic solvent medium, at least one hydrophobic polymer of the polyacrylic and/or polyvinylic type, at least one filler and/or essential oil and at least one keratolytic agent.

The present invention further relates to the use of said solution for the preparation of a cosmetic device for cleansing and care of the skin. The device according to the invention is particularly effective for cleansing the skin instantaneously by the mechanical and chemical removal of impurities which are present on the surface of the skin due to pollution, cell regeneration, perspiration or sebaceous gland secretion.

In one preferred embodiment of the present invention, the solution also contains at least one cosmetically active substance and can be used for the preparation of a device which is intended not only to cleanse but also to treat the skin through instantaneous release of the active substance simultaneously with the cleansing action. The device of the present invention for instantaneous cleansing and care preferably takes the form of a patch.

It is already well known to use, for therapeutic or cosmetic purposes, patches or stamps which consist of a composite structure comprising one or more layers and which are applied to the area of skin to be treated, thereby allowing the release and slow percutaneous diffusion of an active substance present in a reservoir layer. Such patches actually have a first layer called a support layer, which is generally impermeable and occlusive so as to facilitate passage through the skin, a second layer called a reservoir, which is fixed to said first layer and contains the active substance, and also an adhesive layer, either around the periphery or over the whole surface of the patch, protection being assured by a detachable top layer. These patches have to be applied to dry skin and their adhesiveness increases during the period of application, which can range from several hours to several days.

Thus patent FR-A-2 738 744 describes a patch for the controlled release of at least one active compound which is unstable in an oxidizing medium. This patch comprises a support layer to which is fixed an anhydrous reservoir layer consisting of a hydrophobic polymeric matrix of silicone or polyurethane in which particles of an active compound and of at least one water-absorbing agent are dispersed. This type of patch has a low adhesiveness and is a treatment patch with no cleansing power.

U.S. Pat. No. 5,026,552 describes a patch containing a water-swellable polymer which is applied to moist skin for 5 to 10 minutes and is removed in one piece when dry, thereby creating a skin peeling action. The water-swellable polymers described are, inter alia, polyvinyl alcohol, alkali metal salts of crosslinked carboxymethyl cellulose, alkali metal salts of polyacrylic acid, and crosslinked polyalkylene glycols. However, this patch can only transport an active agent through the skin if it is left on overnight, for example.

These cosmetic patches have no adhesiveness at time zero and only become adhesive after moistening and drying.

Patent applications EP-0 514 760 and EP-0 826 364 have furthermore described the use of a composition based on polymers substituted by salifiable functional groups, and on a volatile solvent such as water, ethanol or isopropyl alcohol, for the removal of blackheads. The efficacy of the composition is improved by the additional incorporation of pigments. The constituent polymers of the composition are preferably water-soluble but can also be hydrophobic, in which case they take the form of dispersions or emulsions. The composition is applied to the skin like a poultice with the aid of a cotton, silk, Nylon® or even plastic tissue and is removed when dry. The composition can also contain an oil to give the film formed a degree of strength, thereby avoiding any rupture when it is removed. This composition has to be applied for a relatively long period of at least 30 minutes to create a peeling effect, but does not allow the delivery of an active substance.

International patent application WO 93/05893 has also proposed a composition consisting of polymers with adhesive properties, such as PVP, PVP/vinyl acetate mixtures and methyl vinyl ether/maleic acid mixtures, in a volatile solvent medium. The composition, which takes the form of a liquid or gel, is applied directly to the skin and dries rapidly in 3 to 10 minutes to form an elastic and flexible solid layer, which is removed with a tissue or a piece of adhesive plastic. It is often difficult to remove the layer in one piece, however, because it tends to break easily, resulting in incomplete peeling.

After rigorous selection, it has been found that certain hydrophobic polymers themselves have satisfactory adhesive properties on the skin and that, by taking a colorless solution of these polymers in an organic solvent medium containing at least one filler and at least one keratolytic agent, it is possible, after evaporation of the solvent medium, to obtain cleansing devices, especially patches or stamps, which have an excellent self-adhesiveness.

The present invention therefore relates, by way of a novel industrial product, to a colorless solution containing, in a cosmetically acceptable organic solvent medium, (1) at least one self-adhesive hydrophobic polymer selected from polymers or copolymers of the polyacrylic and/or polyvinylic type, (2) at least one filler and/or essential oil, and (3) at least one keratolytic agent, said polymer having an adhesiveness of between 150 and 800 g/cm² after evaporation of said organic solvent.

Within the framework of the present invention, colorless solution is understood as meaning a solution which does not contain pigments or dyestuffs and which therefore has no particular color or has an off-white or creamy color.

The polymers of the colorless solution according to the present invention have a linear structure and are also non-ionic and non-water-swellable. They preferably have a weight-average molecular weight of between 500,000 and 2,500,000 and preferably of 1,000,000 to 2,000,000.

These polymers are generally present in a proportion of between 20 and 60%, based on the total weight of the solution, and, after evaporation of the organic solvent medium, they have an adhesiveness on the skin of between 150 and 800 g/cm² which is constant over time. The adhesiveness of the polymer or polymers corresponds to the force, exerted perpendicularly to the plane of the adhesive surface, which is necessary to detach it from the skin, so it is advantageous to predetermine the proportion of polymers in the solution as a function of the desired adhesiveness.

The polymers according to the invention are preferably selected from copolymers of the acrylic/vinylic type, especially copolymers consisting of units derived from monomers selected from C₁-C₂₀-alkyl acrylates, vinyl acetate and acrylic acid, more particularly ethylhexyl acrylate, vinyl acetate and acrylic acid.

The cosmetically acceptable organic solvent medium according to the invention is made up e.g. of solvents such as ethyl acetate, ethyl alcohol, isopropyl alcohol and mixtures thereof.

The organic solvent is generally present in a proportion of between 20 and 80% of the total weight of the solution.

Filler is understood as meaning any substance which is capable of modifying the surface of the skin in a very short period of time, bringing softness to the skin. Nylon powders such as the product marketed by ATOCHEM under the name ORGASOL®, and polymethyl methacrylate powders such as the products marketed by WACKHERR under the name COVABEAD®, may be mentioned in particular.

The filler and/or essential oil are generally present in a proportion of between 0.1 and 20% and preferably of between 0.5 and 10% by weight, based on the total weight of the composition.

Keratolytic agent is understood as meaning any agent which is capable of cleansing the skin of its impurities, especially dead cells.

Any keratolytic agent can be used in the solutions of the invention without affecting their nature or their subsequent adhesiveness on the skin.

These agents are well known in the cosmetic industry and examples which may be mentioned are α-hydroxycarboxylic acids such as glycolic acid, lactic acid, tartaric acid, malic acid, citric acid and mandelic acid, β-hydroxycarboxylic acids such as salicylic acid, and also fruit acids, β-ketocarboxylic acids and salts, amides or esters thereof. Kojic acid, retinyl palmitate and vitamin C may also be mentioned among these agents.

Preferably, salicylic acid derivatives such as those described in patent application EP-A-0 378 936 are used in the compositions according to the invention. 5-n-Octanoylsalicylic acid, 5-n-decanoylsalicylic acid and 5-n-dodecanoylsalicylic acid may be mentioned among said derivatives without implying a limitation.

The keratolytic agent is generally present in a proportion of between 0.1 and 15% by weight, based on the total weight of the solution.

In one preferred embodiment, the solution according to the present invention also contains at least one cosmetically active substance in a proportion of between 1 and 15% of the total weight of the solution.

The cosmetically active substances which can be incorporated into the solution according to the present invention can be either of the water-soluble type or of the liposoluble type and are generally selected from the substances conventionally used in the cosmetic industry.

In one advantageous mode of carrying out the invention, the cosmetically active substances are homogeneously dispersed in the solution and can take the form of a solid, especially particles.

Said cosmetically active substances can be selected from those which have a moisturizing, reconstituting, softening, emollient, healing, regenerating, astringent, dulling, soothing, self-tanning, firming, deodorant, depigmenting, lightening, refreshing or nourishing action.

They can also be agents which are capable of absorbing and/or regulating sebum and perspiration, liporegulators, sun protectors, skin conditioners, anti-wrinkle agents, toners, anti-free radical agents, anti-acne agents, disinfectants, anti-ageing agents, vascular protectors, insensitizers, immunomodulators, anti-inflammatories, antibacterials or antifungals and, of course, combinations of one or more of these agents.

Examples of other cosmetically active substances which may be mentioned are chitosan and derivatives thereof, tannin, caffeine, essential oils, collagen, sun filters and hyaluronic acid.

The solution according to the invention can also contain particles of at least one water-absorbing agent, homogeneously dispersed in said solution.

The following may preferably be mentioned among the water-absorbing agents present in the dispersed state in the colorless solution according to the invention: superabsorbent crosslinked polyacrylates with a high swelling ratio in water, such as those marketed by NORSOLOR under the name AQUAKEEP®, polyvinyl alcohol, carboxyvinylic polymers such as those marketed by GOODRICH under the name CARBOPOL®, semisynthetic cellulose derivatives such as carboxymethyl cellulose, starches, biogums such as xanthan gum, guar gum, gum arabic and gum tragacanth, biosaccharides, scleroglucans, casein, phytocolloids such as alginates, carrageenates, agar-agar, gelatin and cotton fibers.

It is very particularly preferred to use superabsorbent crosslinked polyacrylates and cellulose derivatives, whose presence in the dispersed state, after hydration, promotes the availability, on the skin, of the particles of the active compounds present.

The water-absorbing agent as defined above is preferably present in a proportion ranging from about 0.2% to about 20% by weight and more particularly ranging from 0.5% to 10%, based on the total weight of the solution.

The present invention further relates to the use of the solution as described above for the preparation of a self-adhesive cosmetic device for cleansing and care of the skin.

According to the invention, the device can take a variety of forms such as a patch or stamp, a roller or any other appropriate form. The device comprises essentially a support which can be for example in the form of a flexible, elastic or rigid, flat or cylindrical surface of variable thickness.

In a first particular embodiment of the invention, a patch is prepared for immediate use by impregnating or coating a support with an appropriate amount of the colorless solution according to the invention, and then evaporating the solvent medium. The resulting patch can then be applied directly to the skin and is held in place by its self-adhesive properties.

In a second embodiment, a ready-to-use patch is prepared from a support which is coated with the colorless solution according to the invention by the conventional method of preparing patches, and to which a detachable protective covering sheet is affixed after evaporation of the solvent medium. When used, the top protective sheet is removed and the patch is then applied to the skin, where it is held in place by its self-adhesiveness.

In both these embodiments, the patch is left on the skin for a very short time of between about 15 seconds and 20 minutes and preferably of between 15 seconds and 10 minutes. The patch is then removed, enabling the skin to be cleansed mechanically and chemically, in one synergistic action, by virtue of the particular adhesiveness of the polymers and the presence of at least one keratolytic agent.

The support can take the form of sheets or films which are perforated or non-perforated, porous or non-porous and alveolate or non-alveolate, woven or non-woven webs, rollers or any other appropriately shaped objects. Said supports can be either occlusive or non-occlusive.

The support can consist for example of a thermoplastic material selected from high and low density polyethylenes, polypropylenes, polyvinyl chlorides, ethylene/vinyl acetate copolymers, polyesters and polyurethanes, or a composite of such materials.

The support can be of any thickness appropriate to the desired function of the support. The thickness of the support is preferably between 20 μm and about 1.5 mm. Advantageously, the support layer is sufficiently flexible to be able to follow the profile of the skin perfectly and not to make the user feel uncomfortable.

If the support is non-occlusive, it is preferred to use one consisting of paper, a porous or perforated thermoplastic material, a woven material or a perforated non-woven material.

The detachable protective sheet can be for example a silicone-coated sheet of paper or a sheet of thermoplastic material treated e.g. with a varnish so as to render it anti-adhesive. This detachable protective sheet is preferably made of polyethylene.

If the patches are not in a pre-cut form, they can be cut according to an appropriate contour corresponding to the surface of the skin to be treated, for example in the shape of a mask for application to the face, or in any other appropriate shape for application especially to the nose, cheeks, eye contours or forehead. The patches generally have a size of between 0.25 cm² and 500 cm² and preferably of between 1 cm² and 30 cm².

After removal of the detachable protective layer where appropriate, the patches made and cut in this way can be used by application to the surface of the skin to be treated, being held perfectly in place by the self-adhesive properties of the polyvinylic and/or polyacrylic polymers.

The present invention further relates to a cosmetic method for rapid cleansing and treatment of the skin, comprising steps consisting in:

(i) applying, to a support as defined above, a sufficient amount of the solution based on a polyacrylic or polyvinylic polymer in a solvent medium;

(ii) leaving the solvent to evaporate;

(iii) applying the coated support to the parts of the skin to be cleansed and treated, for a period of between 15 seconds and 20 minutes, said support being held in place by self-adhesion; and

(iv) removing the support by one of its ends.

Alternatively, the cleansing and treatment method comprises steps consisting in:

(i) detaching the protective covering sheet from a ready-to-use cosmetic patch for instantaneous cleansing and treatment, as described above;

(ii) applying the patch to the parts of the skin to be cleansed and treated, for a period of between 15 seconds and 20 minutes, said patch being held in place by self-adhesion; and finally

(iii) removing said patch.

The application time is preferably between 15 seconds and 5 minutes.

Several Examples of solutions according to the invention will now be given by way of illustration, together with Examples of patches and their use for cleansing and treatment of the skin.

### EXAMPLES

### Example 1

A colorless solution is prepared which comprises the following by weight:

35% of ethylhexyl acrylate/vinyl acetate/acrylic acid copolymer of MW=1,500,000,

52% of ethyl acetate,

3% of 5-n-octanoylsalicylic acid, and

10% of ORGASOL® marketed by ATOCHEM.

A perforated polythene sheet in the shape of a mask and with a thickness of about 30 μm is impregnated with a sufficient amount of the solution of the above formulation. After evaporation of the ethyl acetate, the resulting patch is applied to the face and is then removed a few minutes later. After removal of the patch, the skin is found to have a smoother and cleaner appearance. Furthermore, the presence of ORGASOL® imparts a natural dull appearance and also affords a pleasant sensation of softness.

### Example 2

A colorless solution is prepared which comprises the following by weight:

31% of ethylhexyl acrylate/vinyl acetate/acrylic acid copolymer of MW=1,500,000,

46% of isopropyl alcohol,

3% of 5-n-octanoylsalicylic acid,

10% of kojic acid, and

10% of ORGASOL® marketed by ATOCHEM.

A patch similar to the one described in Example 1 is prepared from the above solution. This patch affords an effective exfoliation of the skin while at the same time effecting a very satisfactory lightening of the face through the presence of kojic acid.

### Example 3

A colorless solution is prepared which comprises the following by weight:

37% of ethylhexyl acrylate/vinyl acetate/acrylic acid copolymer of MW=1,500,000,

50% of isopropyl alcohol,

3% of 5-n-octanoylsalicylic acid,

0.5% of essential oil of geranium,

0.5% of essential oil of citron, and

9% of ORGASOL® marketed by ATOCHEM.

A patch consisting of a non-woven, perforated and alveolate web is prepared by the method described in Example 1. This patch is applied for about 15 seconds to the part of the skin to be cleansed and is then removed to leave the skin visibly cleaner, more supple and softened through the deposit of ORGASOL® on its surface and the emollient action of the essential oils of geranium and citron.

What is claimed is:

1. A colorless solution for cosmetic use, based on a hydrophobic polymer in an organic solvent medium, which contains, in said cosmetically acceptable organic solvent, (1) at least one self-adhesive hydrophobic polymer selected from polymers or copolymers of the polyacrylic and/or polyvinylic type, (2) at least one filler and/or essential oil, and (3) at least one keratolytic agent, said polymer having an adhesiveness of between 150 and 800 g/cm² after evaporation of said organic solvent.

2. A solution as claimed in claim 1 wherein the polymer consists of units derived from monomers selected from C₁-C₂₀-alkyl acrylates, vinyl acetate and acrylic acid.

3. A solution as claimed in claim 1 wherein the hydrophobic polymer is present in a proportion of between 20 and 60% of the total weight of the solution.

4. A solution as claimed in any one of the preceding claims wherein the keratolytic agent is present in a proportion of between 0.1 and 15% of the total weight of the solution.

5. A solution as claimed in any one of the preceding claims wherein the organic solvent medium is present in a proportion of between 20 and 80% of the total weight of the solution.

6. A solution as claimed in any one of the preceding claims wherein the filler and/or essential oil are present in a proportion of between 0.1 and 20% by weight, based on the total weight of the composition.

7. A solution as claimed in any one of the preceding claims which also contains a cosmetically active substance.

8. A solution as claimed in claim 7 wherein the cosmetically active substance is present in a proportion of between 1 and 15% of the total weight of the solution.

9. A solution as claimed in any one of the preceding claims which also contains a water-absorbing compound.

10. Use of the solution as claimed in any one of the preceding claims for the preparation of a device for instantaneous cleansing and care of the skin.

11. Use as claimed in claim 10 wherein said device is a patch formed of a support which has been coated with the solution as claimed in claims 1 to 9.

12. Use as claimed in claims 10 and 11 wherein said support takes the form of a sheet or a film which is woven or non-woven, perforated or non-perforated, occlusive or non-occlusive, porous or non-porous and alveolate or non-alveolate.

13. Use as claimed in claims 10 and 11 wherein said support takes the form of a cylinder.

14. Use as claimed in any one of claims 10 to 13 wherein said skin cleansing device also comprises a detachable top protective sheet.

15. A patch for instantaneous cleansing and care of the skin, comprising a support impregnated with a sufficient amount of the solution as claimed in claims 1 to 9.

16. A cosmetic method of cleansing the skin, comprising steps consisting in:
(i) applying, to a support, a sufficient amount of the solution as claimed in claims 1 to 9;
(ii) leaving the solvent to evaporate;
(iii) applying the coated support to the parts of the skin to be cleansed, for a period of between about 15 seconds and 20 minutes; and
(iv) removing the support by one of its ends.
